(19) United States
(12) Patent Application Publication
(10) Pub. No.: US 2025/0128001 A1
(43) Pub. Date: Apr. 24, 2025

(54) DEVICES, ASSEMBLIES, AND METHODS FOR DELIVERING POWDERED AGENTS

(71) Applicant: Boston Scientific Medical Device Limited, Galway (IE)

(72) Inventors: Nitesh Ghananil BAVISKAR, Kalyan West (IN); Nabarun BHOWMICK, Kolkata (IN)

(73) Assignee: Boston Scientific Medical Device Limited, Galway (IE)

(21) Appl. No.: 18/915,522

(22) Filed: Oct. 15, 2024

Related U.S. Application Data
(60) Provisional application No. 63/591,166, filed on Oct. 18, 2023.

(51) Int. Cl. A61M 15/00

(52) U.S. Cl. CPC A61M 15/0001; A61M 2202/064

(57) ABSTRACT

A medical delivery device that includes a body, a fluid source configured to store a pressurized fluid, an enclosure configured to store an agent, the enclosure is movably coupled to the body, and a first actuator configured to release the pressurized fluid from the fluid source into the body. The enclosure is configured to move relative to the body between a first position and a second position. The enclosure is fluidly decoupled from the fluid source when in the first position such that the pressurized fluid released into the body is not in fluid communication with the agent stored in the enclosure. The enclosure is fluidly coupled to the fluid source when in the second position such that the pressurized fluid released into the body is in fluid communication with the agent stored in the enclosure.

### CROSS-REFERENCE TO RELATED APPLICATIONS

This application claims the benefit of priority under 35 U.S.C. § 119 from U.S. Provisional Application No. 63/591,166, filed Oct. 18, 2023, which is incorporated by reference herein in its entirety.

### TECHNICAL FIELD

Various aspects of this disclosure relate generally to medical devices for delivering agents. More specifically, in embodiments, this disclosure relates to handheld device assemblies for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a device inserted into the working channel of the endoscope. Agent delivery may be achieved, for example, through the use of pressurized fluid that helps to move the agent through the device and toward the remote site. Such devices, however, may include numerous actuators, complex assemblies, and complicated configurations for loading a pressurized fluid source. Aspects of this disclosure may help to solve one or more of these issues or other issues in the art.

### SUMMARY

Each of the aspects disclosed herein may include one or more of the features described in connection with any of the other disclosed aspects.

According to an example, a medical delivery device includes a body; a fluid source configured to store a pressurized fluid; an enclosure configured to store an agent, the enclosure is movably coupled to the body; and a first actuator configured to release the pressurized fluid from the fluid source into the body; wherein the enclosure is configured to move relative to the body between a first position and a second position, the enclosure is fluidly decoupled from the fluid source when in the first position such that the pressurized fluid released into the body is not in fluid communication with the agent stored in the enclosure, and the enclosure is fluidly coupled to the fluid source when in the second position such that the pressurized fluid released into the body is in fluid communication with the agent stored in the enclosure.

Any of the medical delivery devices described herein may include any of the following features. The first actuator is configured to cause the pressurized fluid to at least partially enter the enclosure when the enclosure is in the second position relative to the body. The first actuator is configured to cause the pressurized fluid to agitate the agent, thereby forming a mixture of the agent and the pressurized fluid. The medical delivery device further includes a delivery conduit fluidly coupled to the body; wherein the first actuator is configured to cause the pressurized fluid to move the mixture out of the body and into the delivery conduit. The first actuator is configured to release the pressurized fluid from the fluid source at a first pressure level. The medical delivery device further includes a second actuator configured to release the pressurized fluid from the fluid source and into the body at a second pressure level that is less than the first pressure level. The second actuator is configured to cause the pressurized fluid to inhibit a buildup of moisture within the delivery conduit without delivering the agent when the enclosure is in the first position. The second actuator is configured to cause the pressurized fluid to flush the delivery conduit of a residual material without delivering the agent when the enclosure is in the first position. The first actuator includes a trigger mechanism that is depressible relative to the body, and wherein the second actuator includes a lever mechanism that is pivotable relative to the body. The body includes a handle having an interior cavity, and a cartridge configured to receive the fluid source; wherein the cartridge is releasably coupled to the handle such that the fluid source is disposed within the interior cavity when the cartridge is coupled to the handle. The cartridge includes a second actuator that is configured to move the fluid source within the interior cavity to fluidly couple the fluid source to the body. The body includes a second actuator along the handle that is configured to move the fluid source within the interior cavity to fluidly couple the fluid source to the body. The body includes a handle having an interior cavity, the handle is movable relative to the body from an open configuration to a closed configuration; wherein, when in the open configuration, the handle is at least partially detached from the body and configured to receive the fluid source within the interior cavity, and in the closed configuration, the handle is attached to the body and configured to fluidly couple the fluid source to the body. The medical delivery device further includes a second actuator configured to move between a plurality of positions relative to the body to control an operating mode of the medical delivery device. In a first position of the plurality of positions, the second actuator is configured to control the medical delivery device in a neutral operating mode such that the pressurized fluid is not released from the fluid source upon actuation of the first actuator; wherein, in a second position of the plurality of positions, the second actuator is configured to control the medical delivery device in a low pressure operating mode such that the pressurized fluid is released from the fluid source at a low pressure level upon actuation of the first actuator; and wherein, in a third position of the plurality of positions, the second actuator is configured to control the medical delivery device in a high pressure operating mode such that the pressurized fluid is released from the fluid source at a high pressure level that is greater than the low pressure level upon actuation of the first actuator.

According to another example, a medical delivery device, includes a body including a handle, a central body, and a nozzle, the body is configured to be fluidly coupled to a fluid source storing a pressurized fluid; an enclosure storing an agent; and an actuator configured to release the pressurized fluid from the fluid source into the body; wherein the enclosure is configured to move relative to the body from a first position, in which the enclosure is fluidly decoupled from the fluid source such that the pressurized fluid released into the body upon actuation of the actuator is inhibited from fluidly communicating with the agent stored in the enclosure, to a second position in which the enclosure is fluidly coupled to the fluid source such that the pressurized fluid released into the body upon actuation of the actuator fluidly communicates with the agent stored in the enclosure.

Any of the medical delivery devices described herein may include any of the following features. When the enclosure is in the first position, the pressurized fluid released from the fluid source into the body is at a first pressure level; and wherein, when the enclosure is in the second position, the pressurized fluid released from the fluid source into the body is at a second pressure level that is greater than the first pressure level. The actuator is configured to cause the pressurized fluid at the first pressure level to inhibit a buildup of moisture within a delivery conduit coupled to the nozzle without delivering the agent out of the body when the enclosure is in the first position. The actuator is configured to cause the pressurized fluid at the second pressure level to agitate the agent and form a mixture of the agent and the pressurized fluid for delivery out of the body when the enclosure is in the second position.

According to another example, a method for delivering an agent from a medical delivery device that includes a body, a fluid source, an enclosure, and an actuator, the method includes moving the enclosure to a first position relative to the body to fluidly decouple the enclosure from the fluid source; actuating the actuator to release a pressurized fluid from the fluid source into the body, wherein the pressurized fluid is not in fluid communication with the agent stored in the enclosure when in the first position; moving the enclosure to a second position relative to the body to fluidly couple the enclosure to the fluid source; and actuating the actuator to release the pressurized fluid from the fluid source into the body, wherein the pressurized fluid is in fluid communication with the agent stored in the enclosure when in the second position; wherein the pressurized fluid agitates the agent and forms a mixture of the agent and the pressurized fluid for delivery out of the medical delivery device.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The terms “top,” “up,” or “upper” refer to a direction or side of a device relative to its orientation during use, and the terms “bottom,” “down,” or “lower” refer to a direction or side of a device relative to its orientation during use that is opposite of the “top,” “up,” or “upper.” The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIGS. 1A-1B show an exemplary delivery device, according to some embodiments.

FIGS. 2A-2B show a partial side view of a cartridge of the delivery device of FIGS. 1A-1B, according to some embodiments.

FIGS. 3A-3B show another exemplary delivery device, according to some embodiments.

FIGS. 4A-4C show another exemplary delivery device, according to some embodiments.

FIG. 5 shows another exemplary delivery device, according to some embodiments.

FIG. 6 shows another exemplary delivery device, according to some embodiments.

FIGS. 7A-7B show another exemplary delivery device, according to some embodiments.

FIG. 8 shows another exemplary delivery device, according to some embodiments.

### DETAILED DESCRIPTION

Embodiments of this disclosure relate to delivery devices used to deliver or dispense various agents (e.g., powdered agents) to treatment sites within subjects (e.g., patients) during certain medical procedures. For example, delivery devices may receive and be fluidly coupled to a pressurized medium source (e.g., a gas canister) from which a pressurized fluid (e.g., a gas) may be released into the delivery device for interacting with an agent housed within the device. The pressurized fluid may mix with the agent to facilitate movement of the agent through the delivery device, and towards an outlet of the device (e.g., a catheter) positioned adjacent to a target treatment site within the subject (e.g., a patient).

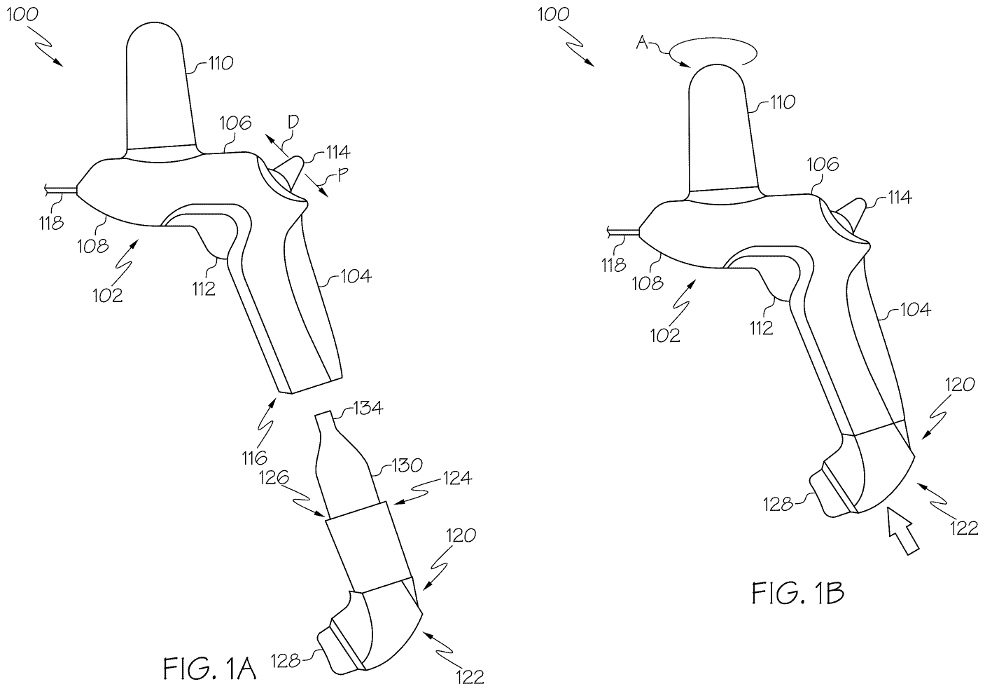

FIGS. 1A-1B show aspects of an exemplary delivery device 100 according to some embodiments. Delivery device 100 may be an agent (e.g., powder) delivery system. Delivery device 100 may include a body 102 having a proximal portion that is sized, shaped, and/or otherwise configured as a handle 104. Delivery device 100 may also include a distal portion that is sized, shaped, and/or otherwise configured as a nozzle 108. Delivery device 100 may further include an intermediate portion that is sized, shaped, and/or otherwise configured as a central body 106 positioned between handle 104 and nozzle 108. In some examples, handle 104 may define an interface for grasping body 102 with a hand of a user during use of delivery device 100. Body 102 may include, or may be configured to receive, an enclosure 110 (or other source or container) storing a material (e.g., a powdered agent). The powdered agent may be, for example, a hemostatic agent. The agent may alternatively be another type of agent or material, or form of agent (e.g., a liquid or gel agent), and may have any desired function without departing from a scope of this disclosure.

Enclosure 110 may be releasably coupled to a portion of body 102 for providing the agent to body 102. In some embodiments, enclosure 110 may be screwed onto or otherwise attached to body 102 for supplying the agent from enclosure 110 to body 102. In some examples, enclosure 110 may be movably coupled to central body 106, however, in other examples, enclosure 110 may be coupled to various other portions of body 102 (e.g., nozzle 108). Enclosure 110 may be configured to move (e.g., rotate in a direction of arrow A) between one or more positions relative to body 102, such as, for example, to transition enclosure 110 between one or more fluid states (e.g., fully open, partially open, closed, etc.). In the example, enclosure 110 may be fluidly coupled to one or more other components of delivery device 100 (e.g., a gas canister 130) for mixing the agent with a material (e.g., a pressurized gas) released from said component based on a position of enclosure 110 relative to body 102.

Still referring to FIGS. 1A-1B, delivery device 100 may include one or more actuators positioned along body 102 at locations that facilitate an ergonomic grasp and control of said actuators. Additionally, the one or more actuators may be sized, shaped, and/or otherwise configured to have a cross-sectional profile that facilitates actuation of the actuators with ease by a hand of a user of delivery device 100. For example, delivery device 100 may include a first actuator 112 and a second actuator 114 each positioned proximate to handle 104 and/or central body 106. First actuator 112 may be positioned along an exterior surface of body 102 that is different from an exterior surface of body 102 that second actuator 114 may be positioned along. For example, first actuator 112 may be positioned along a lower exterior surface of body 102, and second actuator 114 may be positioned along an upper exterior surface of body 102. As shown and described herein, the one or more actuators of delivery device 100 may be positioned along various other suitable locations and/or surfaces of body 102.

In the example, first actuator 112 may be sized and/or shaped as a trigger mechanism, and may be positioned at a location along body 102 (e.g., on handle 104) to facilitate ease of access by an index-finger of the user during use of delivery device 100. Second actuator 114 may be sized and/or shaped as a lever mechanism, and may be positioned at a location along body 102 (e.g., on central body 106) to facilitate ease of access by a thumb of the user during use of delivery device 100. In other examples, one or more of first actuator 112 and/or second actuator 114 may have various other suitable sizes, shapes, and/or configurations without departing from a scope of this disclosure.

Still referring to FIGS. 1A-1B, each of first actuator 112 and second actuator 114 may be selectively actuated or otherwise moved to control delivery of a pressurized fluid into body 102. For example, first actuator 112 may be configured to activate a flow of pressurized fluid (e.g., gas), from a pressurized medium source that is in fluid communication with delivery device 100, at a first (high) pressure level upon depressing first actuator 112. Second actuator 114 may be configured to activate a flow of pressurized fluid from the pressurized medium source at a second (low) pressure level that is relatively less than the first (high) pressure level. In other words, the first (high) pressure level is of a pressurization that is greater than the second (low) pressure level. In the example, the pressurized medium source may include a fluid (gas) source, and particularly a gas canister 130.

As described herein, delivery device 100 may include a regulator disposed within body 102 that is configured and operable to control a pressurization level of the pressurized fluid released from gas canister 130 into body 102 based on the actuation of at least one of first actuator 112 and second actuator 114. In some embodiments, the lever mechanism of second actuator 114 may be pivotable in a first (distal) direction D and/or a second (proximal) direction P relative to body 102 to activate the flow of pressurized flow at the second (low) pressure. In other embodiments, the lever mechanism of second actuator 114 may only be pivotable in a single direction to initiate release of the pressurized fluid from gas canister 130.

The pressurized fluid released from gas canister 130 may be received through delivery device 100, and particularly into body 102, such as through one or more fluid conduits (not shown) within body 102. Based on a state of enclosure 110 (e.g., open, closed, etc.), as determined by a relative position of enclosure 110 to body 102, the pressurized fluid released into body 102 is permitted to travel towards and/or into enclosure 110 to mix with the agent stored therein. In the example, the pressurized fluid released into body 102 may enter enclosure 110 and mix with the agent therein prior to the mixture exiting enclosure 110 and moving through body 102 towards nozzle 108. In other examples, the agent may be released into body 102 from enclosure 110 and mix with the pressurized fluid within body 102, such as in one or more of the fluid conduits in body 102, prior to the mixture exiting body 102 via nozzle 108.

In some embodiments, a first portion of pressurized fluid from gas canister 130 may be configured to agitate and/or mix with the agent, thereby forming the mixture of the agitated agent and the first portion of pressurized fluid. A second portion of the pressurized fluid may be further configured to deliver the mixture of the agitated agent and the first portion of the pressurized fluid out of body 102 and to a target treatment site (e.g., a desired location within a body of a patient), such as via a delivery conduit 118 (e.g., a catheter) that is coupled to body 102 at nozzle 108.

It should be appreciated that second actuator 114 may be configured to deliver the second (low) pressure alone (e.g., without agent). For example, the pressurized fluid may be delivered at the second (low) pressure for purposes of maintaining delivery conduit 118 in a dry-state as delivery conduit 118 is positioned within the subject and at the target treatment site. In this instance, a buildup of moisture within delivery conduit 118 may be inhibited prior to agent delivery. In other examples, the pressurized fluid may be delivered at the second (low) pressure for insufflation purposes. For example, the pressurized fluid may be delivered at the second (low) pressure to help flush the one or more fluid conduits in body 102 and/or delivery conduit 118 of any residual material (e.g., the agent) in isolation of delivering any additional agent from enclosure 110.

Still referring to FIGS. 1A-1B, delivery device 100 may include a cartridge 120 that is configured to selectively attach to body 102, and particularly to handle 104. Cartridge 120 may have a longitudinal length defined between a proximal end 122 and a distal end 124. Cartridge 120 may include a third actuator 128 positioned adjacent to proximal end 122, and an opening 126 adjacent to distal end 124. Opening 126 may be sized, shaped, and/or otherwise configured to at least partially receive gas canister 130 therethrough. In the example, cartridge 120 may be configured to securely receive gas canister 130 with a distal neck 134 of gas canister 130 extending outwardly from (e.g., beyond) distal end 124 via opening 126. Although not shown, handle 104 may define an interior cavity, channel, and/or lumen that is sized, shaped, and/or otherwise configured to receive gas canister 130 and at least a portion of cartridge 120. Body 102 may include an opening 116 along handle 104 for receiving the portion of gas canister 130 and distal end 124 of cartridge 120 into the interior cavity of handle 104 upon coupling cartridge 120 to body 102, as seen in FIG. 1B.

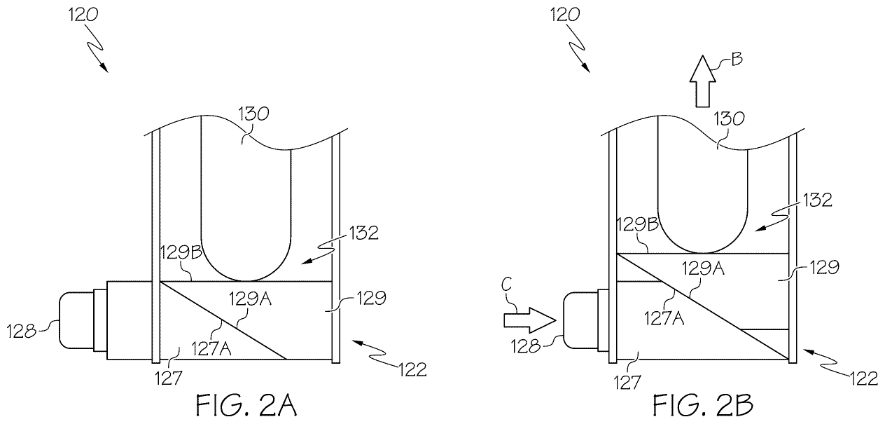

Referring specifically to FIG. 2A, with cartridge 120 coupled to body 102, gas canister 130 may be in a first (unactuated) position in which gas canister 130 is not in fluid communication with the one or more components of delivery device 100 (e.g., the one or more fluid conduits in body 102, enclosure 110, delivery conduit 118, etc.). Cartridge 120 may include a first ramp 127 and a second ramp 129 disposed inside cartridge 120 at proximal end 122. First ramp 127 may have a tapered surface 127A and second ramp 129 may have a corresponding tapered surface 129A that is positioned adjacent to and in contact with tapered surface 127A. In the example, second ramp 129 may be disposed on first ramp 127 with tapered surface 129A abutting against tapered surface 127A. Second ramp 129 may include a non-tapered surface 129B that is positioned adjacent to and in contact with a proximal end 132 of gas canister 130. In the example, gas canister 130 may be disposed on second ramp 129 with proximal end 132 abutting against non-tapered surface 129B.

Referring now to FIG. 2B, first ramp 127 may be movably coupled to third actuator 128 such that movement of third actuator 128 (e.g., in the direction of arrow B) may cause a corresponding movement of first ramp 127. In the example, third actuator 128 may be configured to translate first ramp 127 in a first (lateral) direction relative to second ramp 129 in response to an actuation of third actuator 128. First ramp 127 may be configured to push tapered surface 127A laterally towards tapered surface 129A. Due to a corresponding angled configuration between tapered surface 127A and tapered surface 129A, first ramp 127 may be configured to translate second ramp 129 in a second (vertical) direction relative to first ramp 127 in response to first ramp 127 translating within proximal end 122. In other words, second ramp 129 may be configured to move vertically upwards within proximal end 122 of cartridge 120 in response to a lateral movement of first ramp 127 in proximal end 122. It should be appreciated that the second (vertical) direction of movement for second ramp 129 is relatively transverse (e.g., perpendicular) to the first (lateral) direction of movement for first ramp 127.

Second ramp 129 may be configured to push non-tapered surface 129B vertically upwards towards proximal end 132 of gas canister 130 in response to first ramp 127 translating laterally within proximal end 122, thereby causing gas canister 130 to translate upwards (e.g., in the direction of arrow C) within cartridge 120 in the second (vertical) direction. In this instance, distal neck 134 of gas canister 130 (see FIG. 1A) may move within the interior cavity of handle 104 until interacting with a fluid conduit within body 102 (not shown). For example, the fluid conduit may include a needle and/or pin configured to puncture or pierce a septum/seal of gas canister 130 at distal neck 134. In this instance, gas canister 130 may become fluidly coupled with the fluid conduit in body 102 upon the needle and/or pin puncturing the septum/seal at distal neck 134. In other words, third actuator 128 may be configured to establish fluid communication between gas canister 130 and one or more other components of delivery device 100 (e.g., the one or more fluid conduits in body 102, enclosure 110, delivery conduit 118, etc.) via the fluid conduit (e.g., needle and/or pin) in body 102.

In some embodiments, delivery device 100 may include a regulator (not shown) in body 102 that is fluidly coupled to gas canister 130 upon actuation of third actuator 128. The regulator may be configured to receive the pressurized fluid from gas canister 130 prior to (e.g., upstream of) the other components of delivery device 100, such as enclosure 110 and delivery conduit 118. For example, the needle and/or pin in body 102 may be fluidly coupled to the regulator such that the pressurized fluid released from gas canister 130, upon the needle and/or pin puncturing the seal at distal neck 134, may be initially received at the regulator. The regulator may be configured to receive the pressurized fluid from gas canister 130 at an initial pressurization level and release the pressurized fluid at an adjusted pressurization level, such as at the first (high) pressure and/or the second (low) pressure. In other words, the regulator may be configured to control a pressurization level of the pressurized fluid released to the one or more fluid conduits in body 102, enclosure 110, and/or delivery conduit 118.

In some embodiments, the pressurized fluid may be directed towards or diverted from the regulator based on which of the one or more actuators on deliver device 100 are actuated. For example, the pressurized fluid released from gas canister 130 may be routed towards one or more fluid conduits in body 102 that bypass the regulator upon actuation of first actuator 112, while the pressurized fluid may be routed into the regulator upon actuation of second actuator 114. In other embodiments, the regulator may be omitted entirely.

In exemplary use, with gas canister 130 loaded into cartridge 120, cartridge 120 may be coupled to handle 104 thereby disposing gas canister 130 within the interior cavity of handle 104. Gas canister 130 may be moved into fluid communication with the one or more fluid conduits in body 102 upon actuating third actuator 128. Enclosure 110 may be in a first position relative to body 102 such that enclosure 110 is in a closed fluid state. In this instance, enclosure 110 is fluidly decoupled from gas canister 130, delivery conduit 118, and the one or more fluid conduits of body 102. Although not shown, it should be appreciated that enclosure 110 may include one or more openings (e.g., an inlet opening, an outlet opening, etc.) along a bottom wall of enclosure 110 that interfaces with body 102, and particularly central body 106. The one or more openings of enclosure 110 may be misaligned with corresponding openings on body 102 when enclosure 110 is in the first position.

Delivery device 100 may be actuated via second actuator 114 to deliver pressurized fluid from gas canister 130, through the one or more fluid conduits in body 102 and towards delivery conduit 118, at the second (low) pressure. The second (low) pressure of pressurized fluid delivered into body 102 may be operable for inhibiting moisture buildup in delivery conduit 118 and/or helping to flush residual material (e.g., agent) from the one or more fluid conduits in body 102 and/or delivery conduit 118. With enclosure 110 in the first position, the pressurized fluid is inhibited from interacting with the agent stored in enclosure 110. In other words, the pressurized fluid is not in fluid communication with the agent.

As seen in FIG. 1B, enclosure 110 may be moved (e.g., rotated in the direction of arrow A) to a second position relative to body 102 such that enclosure 110 is transitioned to an open fluid state. For example, the one or more openings of enclosure 110 may be aligned with the corresponding openings on body 102 to fluidly couple enclosure 110 to the one or more components of body 102. In the example, a first opening (e.g., inlet opening) of enclosure 110 may be aligned with a first fluid conduit in body 102 that is in fluid communication with gas canister 130, and a second opening (e.g., outlet opening) of enclosure 110 may be aligned with a second fluid conduit in body 102 that is in fluid communication with delivery conduit 118.

In this instance, enclosure 110 is fluidly coupled with each of gas canister 130 (e.g., via the regulator) and delivery conduit 118 via the one or more fluid conduits of body 102. Delivery device 100 may be actuated via first actuator 112 to deliver pressurized fluid from gas canister 130, through the one or more fluid conduits in body 102, and towards enclosure 110 at the first (high) pressure. The first (high) pressure of pressurized fluid delivered to enclosure 110 may be operable to move (e.g., agitate) the agent from enclosure 110. In some embodiments, the pressurized fluid may agitate the agent within body 102, while in other embodiments, the pressurized fluid may enter enclosure 110 and agitate the agent therein. It should be appreciated that the pressurized fluid at the first (high) pressure may be of a pressurization level sufficient for agitating the agent and delivering a mixture of the agitated agent and pressurized fluid from enclosure 110, through the one or more fluid conduits in body 102, and to the target treatment site via delivery conduit 118. Delivery device 100 may be discarded upon completion of use during a procedure.

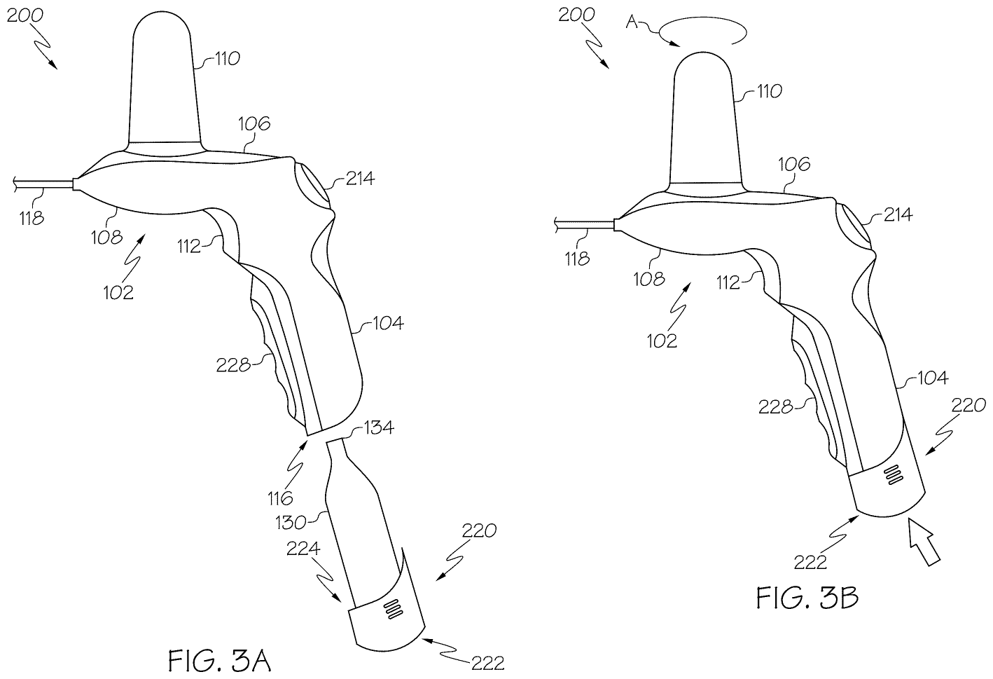

Referring now to FIGS. 3A-3B, aspects of another exemplary delivery device 200 is depicted. Delivery device 200 may be substantially similar to delivery device 100 except for the differences explicitly described herein. Accordingly, the same reference numerals are used to identify substantially similar components. Delivery device 200 may be configured and operable similar to delivery device 100. For example, delivery device 200 may include a cartridge 220 that is configured to selectively attach to body 102, and particularly handle 104. Cartridge 220 may have a longitudinal length defined between a proximal end 222 and a distal end 224. Cartridge 220 may be closed at proximal end 222 and open at distal end 224. Distal end 224 may be sized, shaped, and/or otherwise configured to at least partially receive gas canister 130 therethrough.

In the example, cartridge 220 may be configured to securely receive gas canister 130 with a substantial portion (e.g., more than half) of gas canister 130, including distal neck 134, extending outwardly from (e.g., beyond) distal end 224. Opening 116 along handle 104 may be sized, shaped, and/or otherwise configured to receive at least a portion of gas canister 130 upon coupling cartridge 220 to body 102, as seen in FIG. 3B. In this instance, cartridge 220 is positioned external to the interior cavity of handle 104 upon coupling cartridge 220 to handle 104. With cartridge 220 coupled to handle 104, gas canister 130 may be in a first (unactuated) position in which gas canister 130 is not in fluid communication with the one or more components of delivery device 100.

Still referring to FIGS. 3A-3B, delivery device 200 may include first actuator 112, a second actuator 214, and a third actuator 228. First actuator 112 may be positioned proximate to handle 104 and/or central body 106, and particularly along an exterior lower surface of body 102. First actuator 112 may be configured to activate the flow of pressurized fluid (e.g., gas) from gas canister 130 at the first (high) pressure, upon fluidly coupling gas canister 130 to body 102. Second actuator 214 may be positioned along an upper surface of body 102, and particularly at a location along body 102 (e.g., on central body 106) to facilitate ease of access by a thumb of the user during use of delivery device 200. Second actuator 214 may be sized and/or shaped as a depressible button.

Third actuator 228 may be positioned along an exterior surface of body 102 at a location along handle 104 to facilitate ease of access by one or more fingers of the user during use of delivery device 200. For example, third actuator 228 may extend along a longitudinal length of handle 104, and may be sized, shaped, and/or otherwise configured to receive one or more fingers, for example, multiple fingers of the user as the user grasps handle 104 during use of delivery device 200. Third actuator 228 may be sized and/or shaped as a depressible button. In other examples, one or more of first actuator 112, second actuator 214, and/or third actuator 228 may have various other suitable sizes, shapes, and/or configurations than those shown and described herein without departing from a scope of this disclosure.

Still referring to FIGS. 3A-3B, each of first actuator 112 and second actuator 214 may be selectively actuated (e.g., manually depressible) or otherwise moved to control delivery of a pressurized fluid. For example, first actuator 112 may be configured to activate the flow of pressurized fluid (e.g., gas) from gas canister 130 at the first (high) pressure and second actuator 214 may be configured to activate the flow of pressurized fluid at the second (low) pressure, as described in detail above. Third actuator 228 may be configured to establish fluid communication between gas canister 130 and the one or more components of delivery device 200, such as the one or more fluid conduits in body 102, enclosure 110, and/or delivery conduit 118.

For example, referring specifically to FIG. 3B with cartridge 220 coupled to body 102, gas canister 130 may be in a first (unactuated) position in which gas canister 130 is not in fluid communication with the one or more components of delivery device 200. Third actuator 228 may be configured to move gas canister 130 relative to the interior cavity of handle 104 and/or cartridge 220, such as in an upwards (vertical) direction, to fluidly couple gas canister 130 to the one or more components of delivery device 200. In this instance, distal neck 134 of gas canister 130 (see FIG. 3A) may move within the interior cavity of handle 104 until interacting with the fluid conduit (e.g., needle and/or pin) within body 102 (not shown). In this instance, gas canister 130 may become fluidly coupled with the fluid conduit in body 102.

In exemplary use, gas canister 130 may be loaded into cartridge 220 and cartridge 220 may be coupled to handle 104. Gas canister 130 may be moved into fluid communication with body 102 upon actuating third actuator 228. Enclosure 110 may be in a first position relative to body 102, such that enclosure 110 is in the closed fluid state. In this instance, enclosure 110 is fluidly decoupled from gas canister 130, delivery conduit 118, and the one or more fluid conduits in body 102. Delivery device 200 may be actuated via second actuator 214 to deliver pressurized fluid from gas canister 130, through the one or more fluid conduits in body 102 and towards delivery conduit 118, at the second (low) pressure. The second (low) pressure of pressurized fluid delivered into body 102 may be operable for inhibiting moisture buildup in delivery conduit 118 and/or helping to flush residual material (e.g., agent) from the one or more fluid conduits in body 102 and/or delivery conduit 118.

As seen in FIG. 3B, enclosure 110 may be moved (e.g., rotated in the direction of arrow A) to a second position relative to body 102 such that enclosure 110 is transitioned to the open fluid state. In this instance, enclosure 110 is fluidly coupled with gas canister 130 (e.g., via the regulator), delivery conduit 118, and the one or more fluid conduits in body 102. Delivery device 200 may be actuated via first actuator 112 to deliver pressurized fluid from gas canister 130 and towards enclosure 110 at the first (high) pressure to move (e.g., agitate) the agent stored in enclosure 110. The pressurized fluid may further deliver the mixture of agitated agent and pressurized fluid to the target treatment site from enclosure 110 towards delivery conduit 118 via the one or more fluid conduits in body 102.

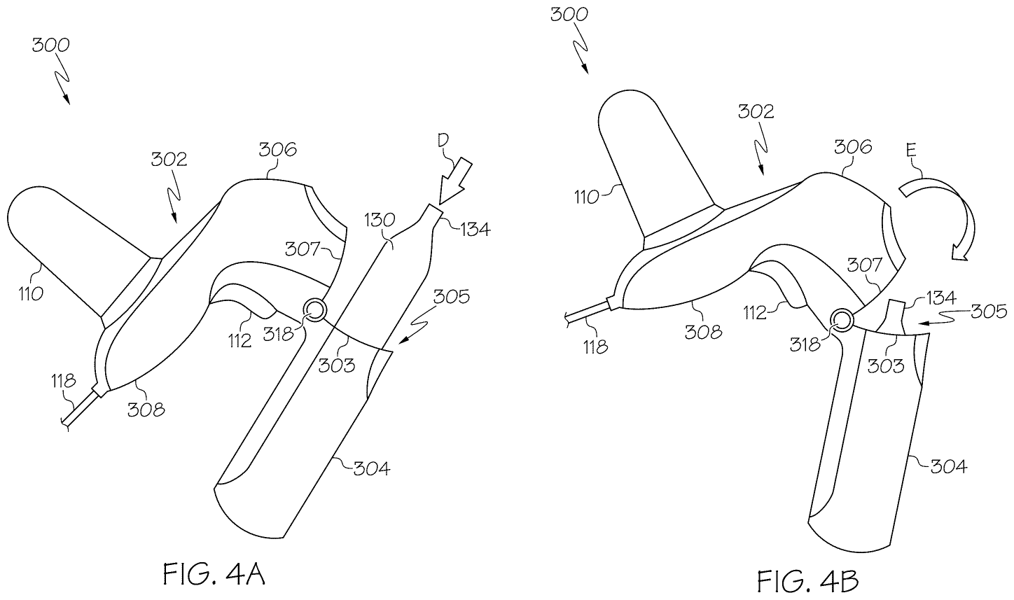

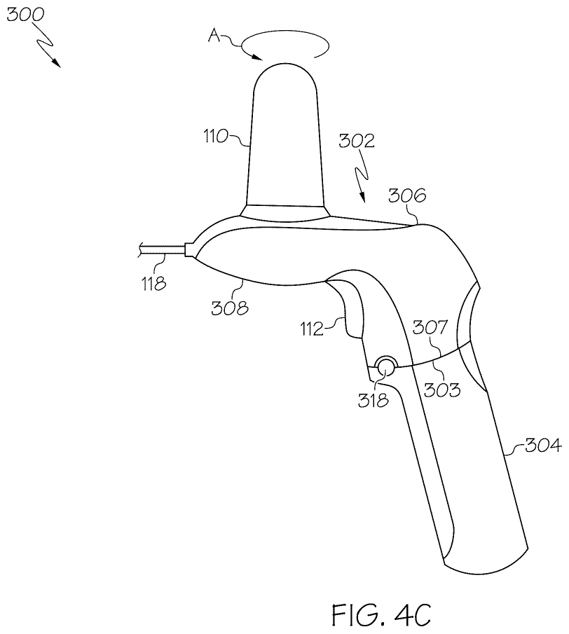

Referring now to FIGS. 4A-4C, aspects of another exemplary delivery device 300 is depicted. Delivery device 300 may be substantially similar to delivery device 100 except for the differences explicitly described herein. Accordingly, the same reference numerals are used to identify substantially similar components. Delivery device 300 may be configured and operable similar to delivery device 100. For example, delivery device 300 may include a body 302 having a proximal portion that is sized, shaped, and/or otherwise configured as a handle 304. Body 302 may also include a distal portion that is sized, shaped, and/or otherwise configured as a nozzle 308, and an intermediate portion that is sized, shaped, and/or otherwise configured as a central body 306 positioned between handle 304 and nozzle 308.

Delivery device 300 may include first actuator 112 positioned proximate to handle 104 and/or central body 106, and particularly along an exterior lower surface of body 302. First actuator 112 may be configured to activate the flow of pressurized fluid from gas canister 130 at the first (high) pressure upon fluidly coupling gas canister 130 to body 302. In the embodiment, delivery device 300 may be inoperable for delivering the second (low) pressure. In other embodiments, first actuator 112 may be further configured to activate a flow of pressurized fluid at the second (low) pressure. In further embodiments, although not shown, delivery device 300 may include another actuator configured to activate the flow of pressurized fluid at the second (low) pressure.

In the example, handle 304 may define an interface for grasping body 302 with a hand of the user during use of delivery device 300. Body 302 may include, or may be configured to receive, enclosure 110. For example, enclosure 110 may be releasably coupled to a portion of body 302 for providing the agent to body 302. In some embodiments, enclosure 110 may be screwed onto or otherwise attached to body 302 for supplying the agent from enclosure 110 to the one or more fluid conduits (not shown) in body 302. In the example, enclosure 110 may be movably coupled to central body 306 and/or nozzle 308, however, in other examples, enclosure 110 may be coupled to various other portions of body 302.

Handle 304 may be movably coupled to central body 306 such that handle 304 may be configured to move between an open configuration (FIG. 4A), a partially-closed configuration (FIG. 4B), and a closed configuration (FIG. 4C). In the example, delivery device 300 may include a second actuator 318 that is configured to unlock and/or release handle 304 from central body 306, thereby allowing handle 304 to move (e.g., pivot) from the closed configuration (FIG. 4C) towards the open configuration (FIG. 4A). In the example, second actuator 318 may include a depressible button. Second actuator 318 may define a pivot point, a hinge, and/or a joint about which handle 304 may move (e.g., pivot in the direction of arrow E) relative to central body 306 between the multiple configurations.

Still referring to FIGS. 4A-4C, handle 304 may define an interior cavity, channel, and/or lumen that is sized, shaped, and/or otherwise configured to receive gas canister 130. Handle 304 may include a distal wall 303 defining an opening 305 that is configured to receive gas canister 130 therethrough (e.g., in the direction of arrow D) for receipt within the interior cavity of handle 304. Central body 306 may include a proximal wall 307 that is sized, shaped, and/or otherwise configured to mate with distal wall 303 when handle 304 is coupled to central body 306 in the closed configuration (see FIG. 4C). As described herein, body 302 may be configured such that handle 304 is locked to central body 306 in the closed configuration upon distal wall 303 contacting proximal wall 307, thereby inhibiting movement of handle 304 and central body 306 relative to one another absent actuation of second actuator 318.

In exemplary use, gas canister 130 may be received within the interior cavity of handle 304 via opening 305 (e.g., in the direction of arrow D) when handle 304 is in the open configuration relative to central body 306, as seen in FIG. 4A. At least a portion of gas canister 130, such as distal neck 134, may extend distally outwards from handle 304 adjacent to distal wall 303 upon fully disposing gas canister 130 in handle 304, as seen in FIG. 4B. In this instance, handle 304 and/or central body 306 may be moved towards each other to transition delivery device 300 to the closed configuration, as seen in FIG. 4C. In particular, handle 304 and/or central body 306 may be pivoted (e.g., in the direction of arrow E) about the pivot point defined by second actuator 318 until distal wall 303 contacts proximal wall 307. It should be appreciated that delivery device 300 may be configured to lock handle 304 to central body 306 upon distal wall 303 and proximal wall 307 contacting one another. In this instance, handle 304 and central body 306 may be decoupled from one another upon actuation of second actuator 318.

In the example, gas canister 130 may be automatically placed into fluid communication with the one or more components of body 302 upon coupling handle 304 to central body 306 in the closed configuration, as seen in FIG. 4C. In other words, distal neck 134 may move with handle 304 towards central body 306 and interact with the fluid conduit (e.g., needle and/or pin) within body 302 as handle 304 is coupled to central body 306. In this instance, gas canister 130 may be fluidly coupled with the fluid conduit in body 302 without requiring further movement of gas canister 130 relative to the interior cavity of handle 304.

Enclosure 110 may be in a first position relative to body 302, such that enclosure 110 is in a closed fluid state. In this instance, enclosure 110 is fluidly decoupled from gas canister 130, delivery conduit 118, and the one or more fluid conduits in body 302. Delivery device 300 may be actuated via first actuator 112 to deliver pressurized fluid from gas canister 130, through the one or more fluid conduits in body 302 and towards delivery conduit 118, to help inhibit moisture buildup in delivery conduit 118 and/or to help flush residual material (e.g., agent) from the one or more fluid conduits in body 302 and/or delivery conduit 118.

Still referring to FIG. 4C, enclosure 110 may be moved (e.g., rotated in the direction A) to a second position relative to body 302 to transition enclosure 110 to the open fluid state. In this instance, enclosure 110 is fluidly coupled with gas canister 130 (e.g., via the regulator), delivery conduit 118, and the one or more fluid conduits in body 302. Delivery device 300 may be further actuated via first actuator 112 to deliver pressurized fluid from gas canister 130, through the one or more fluid conduits in body 302, and towards enclosure 110 to move (e.g., agitate) the agent and deliver the mixture of agitated agent and pressurized fluid to the target treatment site via delivery conduit 118.

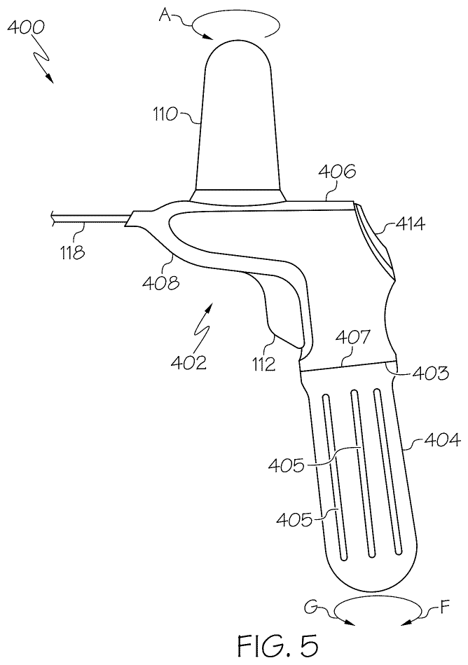

Referring now to FIG. 5, aspects of another exemplary delivery device 400 is depicted. Delivery device 400 may be substantially similar to delivery device 100 except for the differences explicitly described herein. Accordingly, the same reference numerals are used to identify substantially similar components. Delivery device 400 may be configured and operable similar to delivery device 100. For example, delivery device 400 may include a body 402 having a proximal portion that is sized, shaped, and/or otherwise configured as a handle 404. Body 402 may also include a distal portion that is sized, shaped, and/or otherwise configured as a nozzle 408, and an intermediate portion that is sized, shaped, and/or otherwise configured as a central body 406 positioned between handle 404 and nozzle 408.

Delivery device 400 may include first actuator 112 positioned proximate to central body 406, and particularly along an exterior lower surface of body 402. First actuator 112 may be configured to activate the flow of pressurized fluid (e.g., gas) from gas canister 130 at the first (high) pressure, upon fluidly coupling gas canister 130 to body 402. Second actuator 414 may be positioned along an exterior upper surface of body 402 along central body 406. In the example, second actuator 414 may be sized and/or shaped as a depressible button, and positioned at a location along body 402 (e.g., central body 406) to facilitate ease of access by a thumb of the user during use of delivery device 400. Second actuator 414 may be configured to activate the flow of pressurized fluid from gas canister 130 at the second (low) pressure.

Still referring to FIG. 5, handle 404 may be movable relative to central body 406 for selectively coupling handle 404 to central body 406 from an open configuration (not shown) to a closed configuration (FIG. 5). In the example, handle 404 may be configured to detach from central body 406 in response to moving (e.g., rotating in the direction of arrow F) handle 404 in a first direction (e.g., clockwise or counterclockwise) relative to central body 406, and attach to central body 406 in response to moving in a second direction that is opposite of the first direction. For example, handle 404 may be threadably coupled to central body 406. It should be appreciated that, when in the open configuration, handle 404 may be separated from central body 406. Handle 404 may define an interior cavity, channel, and/or lumen that is sized, shaped, and/or otherwise configured to receive gas canister 130 therein.

Handle 404 may include a distal wall 403 defining an opening (not shown) that is configured to receive gas canister 130 therethrough for receipt within the interior cavity of handle 404. Central body 406 may include a proximal wall 407 that is sized, shaped, and/or otherwise configured to mate with distal wall 403 when handle 404 is coupled to central body 406. For example, handle 404 may include an interior threaded portion adjacent to distal wall 403 and central body 406 may include a corresponding interior threaded portion adjacent to proximal wall 407. The interior threaded portion of handle 404 may be configured to mate with the corresponding interior threaded portion of central body 406 when distal wall 403 is positioned in contact with proximal wall 407.

In other embodiments, handle 404 may be configured to selectively couple with central body 406 upon applying a force and/or pushing distal wall 403 against proximal wall 407, thereby snapping handle 404 to central body 406. As described herein, body 402 may be configured such that handle 404 is locked to central body 406 upon distal wall 403 contacting proximal wall 407 when handle 404 is moved to the closed configuration shown in FIG. 5. In this instance, body 402 may be configured to inhibit movement of handle 404 and central body 406 relative to one another absent application of a rotational force to decouple handle 404 from central body 406.

Still referring to FIG. 5, handle 404 may include one or more openings 405 along an exterior surface of handle 404. The one or more openings 405 may be sized, shaped, and/or otherwise configured to maintain the interior cavity of handle 404 at an atmospheric pressure. In the example, openings 405 may be disposed in an array, for example, spaced apart about an outer circumference of handle 404. Openings 405 may be sized and/or shaped as elongated slots extending along a longitudinal length of handle 404. In other examples, openings 405 may have various other suitable shapes, sizes, and/or quantities than those shown and described herein without departing from a scope of this disclosure.

In exemplary use, gas canister 130 may be received within the interior cavity of handle 404. It should be appreciated that at least a portion of gas canister 130, such as distal neck 134, may extend distally outwards from (e.g., beyond) handle 404 adjacent to distal wall 403 upon fully disposing gas canister 130 in handle 404. In this instance, handle 404 and/or central body 406 may be moved (e.g., rotated in the direction of arrow F) relative to one other in the first direction to transition handle 404 to the closed configuration shown in FIG. 5.

It should be appreciated that delivery device 400 may be configured to lock handle 404 to central body 406 upon distal wall 403 and proximal wall 407 contacting one another. In these aspects, handle 404 and central body 406 may only be decoupled from one another upon moving (e.g., rotating in the direction of arrow G) handle 404 and central body 406 relative to one another in the second direction that is opposite of the first direction. In the example, gas canister 130 may be automatically placed into fluid communication with the one or more components of body 402 upon coupling handle 404 to central body 406. In other words, distal neck 134 may move with handle 404 towards central body 406 and interact with the fluid conduit (e.g., needle and/or pin) within body 402 as handle 404 is coupled to central body 406. In this instance, gas canister 130 may be fluidly coupled with the fluid conduit in body 402.

Enclosure 110 may be in a first position relative to body 402 such that enclosure 110 is in the closed fluid state. In this instance, enclosure 110 is fluidly decoupled from gas canister 130, delivery conduit 118, and the one or more fluid conduits in body 402. Delivery device 400 may be actuated via second actuator 414 to deliver pressurized fluid from gas canister 130, through the one or more fluid conduits in body 402 and towards delivery conduit 118, at the second (low) pressure. The second (low) pressure of pressurized fluid delivered through delivery device 400 may be operable for inhibiting moisture buildup in delivery conduit 118 and/or helping to flush residual material (e.g., agent) from the one or more fluid conduits in body 402 and/or delivery conduit 118.

Still referring to FIG. 5, enclosure 110 may be moved (e.g., rotated in the direction of arrow A) to a second position relative to body 402 such that enclosure 110 is transitioned to the open fluid state. In this instance, enclosure 110 is fluidly coupled with gas canister 130 (e.g., via the regulator), delivery conduit 118, and the one or more fluid conduits in body 402. Delivery device 400 may be actuated via first actuator 112 to deliver pressurized fluid from gas canister 130 to enclosure 110 at the first (high) pressure to move (e.g., agitate) the agent and deliver the mixture of agitated agent and pressurized fluid to the target treatment site via delivery conduit 118.

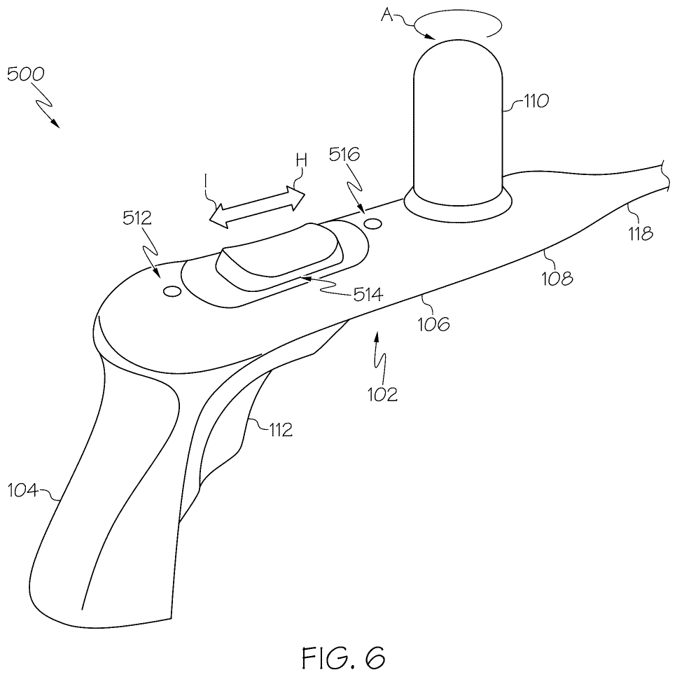

Referring now to FIG. 6, aspects of another exemplary delivery device 500 is depicted. Delivery device 500 may be substantially similar to delivery device 100 except for the differences explicitly described herein. Accordingly, the same reference numerals are used to identify substantially similar components. Delivery device 500 may be configured and operable similar to delivery device 100. For example, although not shown, delivery device 500 may include a pressurized medium source (e.g., gas canister 130) disposed within body 102, such as within one or more of handle 104 and/or central body 106. Delivery device 500 may include first actuator 112 positioned proximate to handle 104, and particularly along an exterior lower surface of body 102. First actuator 112 may be configured to activate the flow of pressurized fluid from gas canister 130.

Delivery device 500 may include a second actuator 514 positioned proximate to central body 106, and particularly along an exterior upper surface of body 102. Second actuator 514 may be sized and/or shaped as a slider mechanism, and positioned at a location along body 102 (e.g., central body 106) to facilitate ease of access by a thumb of the user during use of delivery device 500. Second actuator 514 may be configured to move (e.g., translate) relative to central body 106 between at least two positions, for example, a plurality of positions. For example, second actuator 514 may be configured to translate distally relative to central body 106 to a first (distalmost) position (e.g., in the direction of arrow H) to activate a high pressure mode of delivery device 500 in which actuation of first actuator 112 delivers pressurized fluid at the first (high) pressure.

Still referring to FIG. 6, second actuator 514 may be configured to translate proximally relative to central body 106 to a second (proximalmost) position (e.g., in the direction of arrow I) to activate a low pressure mode of delivery device 500 in which actuation of first actuator 112 delivers pressurized fluid from gas canister 130 at the second (low) pressure. Second actuator 514 may be further configured to translate to a third (intermediate) position located between the first position and the second position to activate a neutral (off) mode of delivery device 500, as seen in FIG. 6. In some embodiments, delivery device 500 may include a user interface and/or display defined by one or more indicators 512, 516 positioned adjacent to second actuator 514, such as along the exterior upper surface of central body 106. The one or more indicators 512, 516 may be configured to facilitate a visual identification of the first position and the second position of second actuator 514. In further embodiments, delivery device 500 may be configured to generate to facilitate a tactile or audible indication and/or feedback of the first position and the second position of second actuator 514.

For example, at least one indicator 512 may be positioned along central body 106 adjacent to the second (proximalmost) position of second actuator 514 and at least one indicator 516 may be positioned along central body 106 adjacent to the first (distalmost) position of second actuator 514. In some embodiments, indicators 512, 516 may include lighting devices configured to emit light when second actuator 514 is positioned at the corresponding position. In other embodiments, indicators 512, 516 may include other indicia to identify the respective operating modes of delivery device 500 when second actuator 514 is moved towards the corresponding indicator 512, 516.

In exemplary use, delivery device 500 may be transitioned from the neutral (off) mode to the low pressure mode by moving (e.g., translating in the direction of arrow I) second actuator 514 from the third (intermediate) position (FIG. 6) to the second (proximalmost) position relative to central body 106. First indicator 512 may be illuminated to indicate to the user of delivery device 500 that the low pressure mode is activated. In this instance, pressurized fluid from gas canister 130 (disposed within body 102) may be delivered at the second (low) pressure upon actuation of first actuator 112. Enclosure 110 may be in a first position relative to body 102, such that enclosure 110 is in the closed fluid state. In this instance, enclosure 110 is fluidly decoupled from gas canister 130, delivery conduit 118, and the one or more fluid conduits in body 102.

Accordingly, the pressurized fluid delivered from gas canister 130 and towards delivery conduit 118 at the second (low) pressure is not in fluid communication with the agent stored in enclosure 110. The second (low) pressure of pressurized fluid delivered through delivery device 500 may be operable for inhibiting moisture buildup in delivery conduit 118 and/or helping to flush residual material (e.g., agent) from the one or more fluid conduits in body 102 and/or delivery conduit 118.

Still referring to FIG. 6, enclosure 110 may be moved (e.g., rotated in the direction of arrow A) to a second position relative to body 102 such that enclosure 110 is transitioned to the open fluid state. In this instance, enclosure 110 is fluidly coupled with gas canister 130 (e.g., via the regulator), delivery conduit 118, and the one or more fluid conduits in body 102. Delivery device 500 may be transitioned to the high pressure mode in response to moving (e.g., translating in the direction of arrow H) second actuator 514 to the first (distalmost) position relative to central body 106. Second indicator 516 may be illuminated to indicate to the user of delivery device 500 that the high pressure mode is activated.

In this instance, pressurized fluid from gas canister 130 (disposed within body 102) may be delivered at the first (high) pressure upon actuation of first actuator 112 to move (e.g., agitate) the agent and deliver the mixture of agitated agent and pressurized fluid to the target treatment site via delivery conduit 118. Upon completion, second actuator 514 may be moved (e.g., translated) to the third (intermediate) position relative to central body 106 to transition delivery device 500 to the neutral (off) mode. Neither of first indicator 512 and second indicator 516 may be illuminated to indicate to the user of delivery device 500 that the neutral (off) mode is activated.

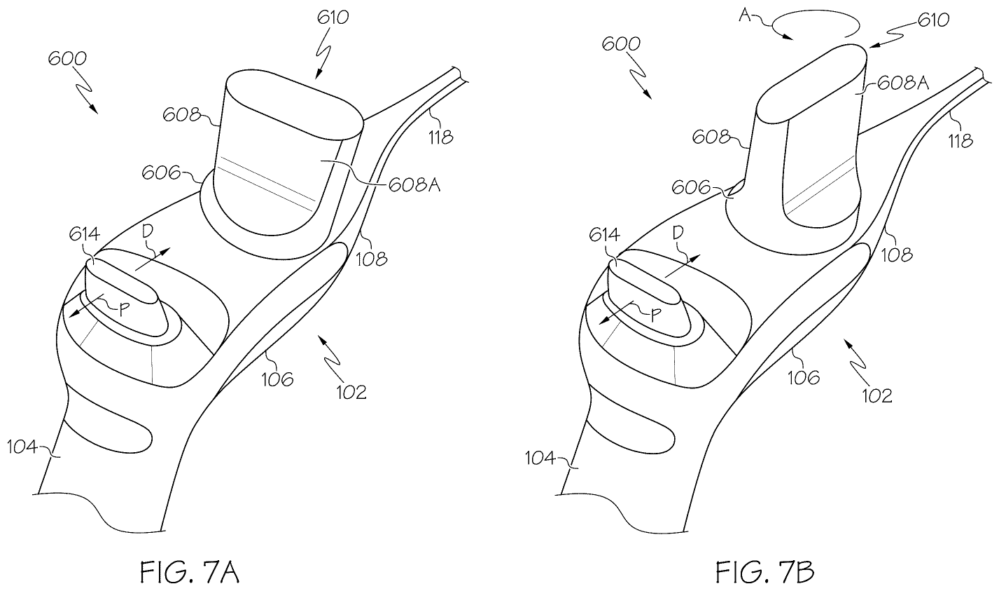

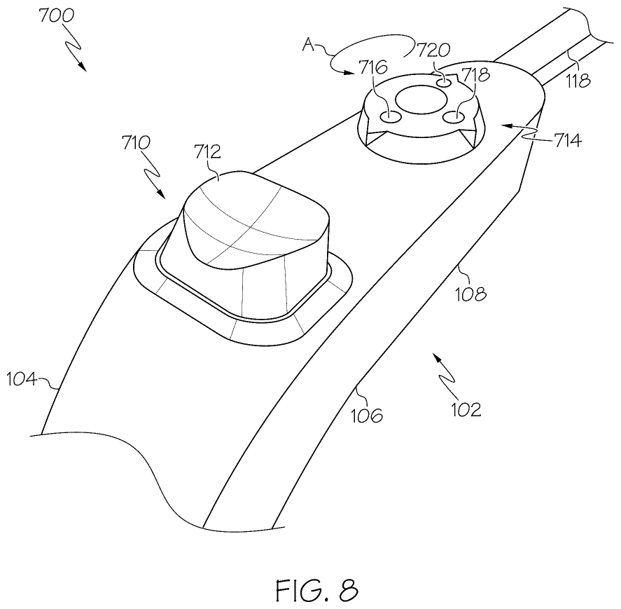

Referring now to FIGS. 7A-7B, aspects of another exemplary delivery device 600 is depicted. Delivery device 600 may be substantially similar to delivery device 100 except for the differences explicitly described herein. Accordingly, the same reference numerals are used to identify substantially similar components. Delivery device 600 may be configured and operable similar to delivery device 100. For example, although not shown, delivery device 600 may include a pressurized medium source (e.g., gas canister 130) disposed within body 102, such as within one or more of handle 104 and/or central body 106.

Delivery device 600 may include an actuator 614 positioned proximate to handle 104 and/or central body 106, and particularly along an exterior upper surface of body 102. Actuator 614 may be configured to activate the flow of pressurized fluid from the gas canister (e.g., gas canister 130). Actuator 614 may be sized and/or shaped as a lever mechanism, and positioned at a location along body 102 (e.g., central body 106) to facilitate ease of access by a thumb of the user during use of delivery device 600. In some embodiments, the lever mechanism of actuator 614 may be pivotable in a first (distal) direction (e.g., in the direction of arrow D) and/or a second (proximal) direction (e.g., in the direction of arrow P) relative to body 102 to activate the flow of pressurized flow. In other embodiments, the lever mechanism of actuator 614 may only be pivotable in a single direction.

Still referring to FIGS. 7A-7B, delivery device 600 may include an enclosure 610 that is releasably coupled to a portion of body 102 for providing the agent to body 102. In some embodiments, enclosure 610 may be screwed onto or otherwise attached to body 102 for supplying the agent from enclosure 610 to the one or more fluid conduits in body 102. In the example, enclosure 610 may be movably coupled to central body 106 and/or nozzle 108, however, in other examples enclosure 610 may be coupled to various other portions of body 102. Enclosure 610 may be sized and/or shaped to form a cross-sectional profile that facilitates manually grasping enclosure 610. For example, enclosure 610 may have an upper body 608 that is narrowed and/or tapered relative to a lower body 606 that is coupled directly to body 102. In the example, upper body 608 may include one or more planar and/or flat surfaces 608A defining the narrowed and/or tapered profile of upper body 608. Flat surfaces 608A may be configured to improve grasping enclosure 610 when moving (e.g., rotating in the direction of arrow A) enclosure 610 during use of delivery deice 600, as described in detail herein.

Enclosure 610 may be configured to move (e.g., rotate in the direction of arrow A) between one or more positions relative to body 102, such as, for example, to transition enclosure 610 between one or more fluid states (e.g., open, closed, etc.). In the example, enclosure 610 may be fluidly coupled to the gas canister (disposed in body 102) for mixing the agent stored in enclosure 610 with the pressurized fluid released from the gas canister based on a position of enclosure 610 relative to body 102. In the embodiment, actuator 614 may be configured to deliver pressurized fluid at a fixed (constant) pressure (e.g., high pressure) irrespective of the position of enclosure 610 relative to body 102. In other embodiments, enclosure 610 may be configured to transition an operating mode of delivery device 600 (e.g., a low operating mode, a high operating mode, etc.) based on a position of enclosure 610 relative to body 102 for delivering pressurized fluid at varying pressure levels (e.g., a low pressure level, a high pressure level, etc.) upon actuation actuator 614.

Referring specifically to FIG. 7A, enclosure 610 may be configured to set delivery device 600 to a low pressure mode when in a first position relative to body 102, such that actuation of actuator 114 activates a flow of pressurized fluid at a low pressure. In this instance, pressurized fluid at the low pressure may be delivered through the one or more fluid conduits in body 102 and towards delivery conduit 118 without mixing with the agent due to enclosure 610 being positioned in the closed fluid state. Enclosure 610 may be further configured to set delivery device 600 to a high pressure mode when moved (e.g., rotated in the direction of arrow A) to a second position relative to body 102, as seen in FIG. 7B. In this instance, actuation of actuator 114 activates a flow of pressurized fluid at a high pressure and the pressurized fluid at the high pressure may be delivered through the one or more fluid conduits in body 102 and towards enclosure 610 for mixing with the agent due to enclosure 610 being positioned in the open fluid state.

In some embodiments, enclosure 610 may be configured to change the operating mode of delivery device 600 (e.g., low pressure mode, high pressure mode, etc.) and/or the fluid state of enclosure 610 (e.g., closed fluid state, open fluid state, etc.) upon moving (e.g., rotating in the direction of arrow A) enclosure 610 by a predetermined distance relative to body 102. For example, the predetermined distance of movement for transitioning delivery device 600 between different operating modes and/or enclosure 610 between different fluid states may be about 90 degrees.

In exemplary use, enclosure 610 may be in a first position relative to body 102 such that enclosure 610 is in the closed fluid state, as seen in FIG. 7A. In this instance, enclosure 610 is fluidly decoupled from the gas canister, delivery conduit 118, and the one or more fluid conduits in body 102. Accordingly, the pressurized fluid delivered from the gas canister and towards delivery conduit 118, in response to moving (e.g., pivoting in the direction of arrow P) actuator 614 towards the second (proximal) position, is not in fluid communication with the agent stored in enclosure 610. In other embodiments, in addition to maintaining enclosure 610 in the closed fluid state, enclosure 610 may be configured to activate a low pressure mode of delivery device 600 when in the first position. In this instance, actuation of actuator 614 provides delivery of pressurized fluid at the low pressure. The low pressure level of pressurized fluid delivered through delivery device 600 may be operable for inhibiting moisture buildup in delivery conduit 118 and/or helping to flush residual material (e.g., agent) from the one or more fluid conduits in body 102 and/or delivery conduit 118.

Referring to FIG. 7B, enclosure 610 may be moved (e.g., rotated in the direction of arrow A) to a second position relative to body 102 by manually grasping the narrowed and/or tapered profile of upper body 608, and particularly the one or more flat surfaces 608A, such that enclosure 610 is transitioned to the open fluid state. In this instance, enclosure 610 is fluidly coupled with the gas canister (e.g., via the regulator), delivery conduit 118, and the one or more fluid conduits in body 102. In some embodiments, delivery device 600 may also be transitioned to the high pressure mode in response to moving (e.g., rotating in the direction of arrow A) enclosure 610 to the second position relative to body 102. In this instance, pressurized fluid from the gas canister (disposed within body 102) may be delivered at the high pressure upon actuation of actuator 614 (e.g., moving in the direction of arrow D to the first position) to move (e.g., agitate) the agent and deliver the mixture of agitated agent and pressurized fluid to the target treatment site via delivery conduit 118.

Referring now to FIG. 8, aspects of another exemplary delivery device 700 is depicted. Delivery device 700 may be substantially similar to delivery device 100 except for the differences explicitly described herein. Accordingly, the same reference numerals are used to identify substantially similar components. Delivery device 700 may be configured and operable similar to delivery device 100. For example, delivery device 700 may include a pressurized medium source (e.g., gas canister 130) disposed within body 102, such as within one or more of handle 104 and/or central body 106.

Delivery device 700 may include an enclosure 710 having an upper surface 712 that is movable (e.g., depressible) relative to body 102. Enclosure 710 may be positioned proximate to handle 104 and/or central body 106, and particularly along an exterior upper surface of body 102. Enclosure 710 may be configured to activate the flow of pressurized fluid (e.g., gas) from the gas canister upon actuating (e.g., moving) upper surface 712 relative to body 102. Upper surface 712 may be sized and/or shaped as a depressible button, and positioned at a location along body 102 (e.g., handle 104 and/or central body 106) to facilitate ease of access by a thumb of the user during use of delivery device 700. It should be appreciated that actuation (e.g., movement) of upper surface 712 causes a corresponding movement of enclosure 710, such that enclosure 710 is simultaneously depressible relative to body 102.

Still referring to FIG. 8, delivery device 700 may include an actuator 714 positioned along body 102, and particularly along the exterior upper surface of nozzle 108. Actuator 714 may be sized and/or shaped as a dial that is movable (e.g., rotatable in the direction of arrow A) relative to nozzle 108 for transitioning delivery device 700 between multiple operating modes. For example, actuator 714 may be configured to move (e.g., rotate) relative to nozzle 108 between a plurality of positions and/or orientations, with each position corresponding to a different operating mode of delivery device 700. For example, actuator 714 may be configured to rotate relative to nozzle 108 to a first position to activate a neutral (off) mode of delivery device 700 in which actuation of upper surface 712 of enclosure 710 does not deliver pressurized fluid from the gas canister to the one or more fluid conduits in body 102.

Actuator 714 may be configured to rotate relative to nozzle 108 (e.g., in the direction of arrow A) to a second position to activate a low pressure mode of delivery device 700 in which actuation of upper surface 712 delivers pressurized fluid from the gas canister at the second (low) pressure. Actuator 714 may be further configured to rotate relative to nozzle 108 (e.g., in the direction of arrow A) to a third position to activate a high pressure mode of delivery device 700 in which actuation of upper surface 712 delivers pressurized fluid at the first (high) pressure.

In some embodiments, delivery device 700 may include a user interface and/or display defined by one or more indicators 716, 718, 720 positioned along actuator 714. The one or more indicators 716, 718, 720 may be configured to facilitate a visual identification of the respective positions of actuator 714 relative to nozzle 108 and the corresponding operating mode of delivery device 700. For example, a first indicator 716 may be positioned along actuator 714 indicative of a neutral (off) mode of delivery device 700, such that actuator 714 may be configured to transition delivery device 700 to the neutral (off) mode when actuator 714 is rotated to the first position (e.g., in the direction of arrow A) with first indicator 716 facing distally towards delivery conduit 118.

Still referring to FIG. 8, a second indicator 718 may be positioned along actuator 714 indicative of the low pressure mode of delivery device 700, such that actuator 714 may be configured to transition delivery device 700 to the low pressure mode when actuator 714 is rotated to the second position (e.g., in the direction of arrow A) with second indicator 718 facing distally towards delivery conduit 118. A third indicator 720 may be positioned along actuator 714 indicative of the high pressure mode of delivery device 700, such that actuator 714 may be configured to transition delivery device 700 to the high pressure mode when actuator 714 is rotated to the third position (e.g., in the direction of arrow A) with third indicator 720 facing distally towards delivery conduit 118, as shown in FIG. 8.

It should be appreciated that the respective indicators 716, 718, 720 may be positioned at various other suitable locations along actuator 714 than that shown and described herein without departing from a scope of this disclosure. Additionally and/or alternatively, actuator 714 may be configured and operable to be positioned and/or facing various other suitable directions relative to body 102 for transitioning delivery device 700 to a different operating mode than the distal direction (facing delivery conduit 118) shown and described herein without departing from a scope of this disclosure. For example, actuator 714 may be configured to face proximally away from delivery conduit 118 and/or laterally relative delivery conduit 118 for activating the corresponding operating mode of delivery device 700.

In some embodiments, indicators 716, 718, 720 may include lighting devices configured to emit light when actuator 714 is positioned at the corresponding position. In other embodiments, indicators 716, 718, 720 may include other indicia (e.g., colors, symbols, letters, etc.) to identify the respective operating modes of delivery device 700 when actuator 714 is moved such that the corresponding indicator 716, 718, 720 is positioned facing distally towards delivery conduit 118. In further embodiments, delivery device 700 may be configured to generate to facilitate a tactile or audible indication and/or feedback of the first, second, and third positions of actuator 714, respectively.

In exemplary use, delivery device 700 may be transitioned from the neutral (off) mode to the low pressure mode by moving (e.g., rotating in the direction of arrow A) actuator 714 from the first position, in which first indicator 716 faces distally towards delivery conduit 118, to the second position in which second indicator 718 faces distally towards delivery conduit 118. In this instance, pressurized fluid from the gas canister (disposed within body 102) may be delivered at the second (low) pressure upon actuation of upper surface 712. Enclosure 710 may be moved simultaneously upon actuation of upper surface 712 from a first position to a second position relative to body 102.

When in the low pressure mode, enclosure 710 remains in the closed fluid state in each of the first position and the second position. Accordingly, enclosure 710 is fluidly decoupled from gas canister 130, delivery conduit 118, and the one or more fluid conduits in body 102. In this instance, the pressurized fluid delivered from the gas canister and towards delivery conduit 118 at the second (low) pressure is not in fluid communication with the agent stored in enclosure 110. The second (low) pressure of pressurized fluid delivered through delivery device 700 may be operable for inhibiting moisture buildup in delivery conduit 118 and/or helping to flush residual material (e.g., agent) from the one or more fluid conduits in body 102 and/or delivery conduit 118.

Still referring to FIG. 8, delivery device 700 may be transitioned to the high pressure mode in response to moving (e.g., rotating in the direction of arrow A) actuator 714 from the second position, in which second indicator 718 faces distally towards delivery conduit 118, to the third position in which third indicator 720 faces distally towards delivery conduit 118. In this instance, pressurized fluid from the gas canister (disposed within body 102) may be delivered at the first (high) pressure upon actuation of upper surface 712. Enclosure 710 may be moved upon actuation of upper surface 712 from the first position to the second position relative to body 102. When in the high pressure mode, enclosure 710 is transitioned from the closed fluid state to the open fluid state in response to moving from the first position to the second position. Accordingly, enclosure 710 is fluidly coupled to the gas canister (e.g., via the regulator), delivery conduit 118, and the one or more fluid conduits in body 102.

In this instance, the pressurized fluid delivered from the gas canister at the first (high) pressure is in fluid communication with the agent stored in enclosure 110. The first (high) pressure of pressurized fluid delivered through delivery device 700 may be operable to move (e.g., agitate) the agent and deliver the mixture of agitated agent and pressurized fluid to the target treatment site via delivery conduit 118. Upon completion, actuator 714 may be moved (e.g., rotated in the direction of arrow A) back to the first position to return delivery device 700 to the neutral (off) mode.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical delivery device, comprising:
a body;
a fluid source configured to store a pressurized fluid;
an enclosure configured to store an agent, the enclosure is movably coupled to the body; and
a first actuator configured to release the pressurized fluid from the fluid source into the body;
wherein the enclosure is configured to move relative to the body between a first position and a second position, the enclosure is fluidly decoupled from the fluid source when in the first position such that the pressurized fluid released into the body is not in fluid communication with the agent stored in the enclosure, and the enclosure is fluidly coupled to the fluid source when in the second position such that the pressurized fluid released into the body is in fluid communication with the agent stored in the enclosure.

2. The medical delivery device of claim 1, wherein the first actuator is configured to cause the pressurized fluid to at least partially enter the enclosure when the enclosure is in the second position relative to the body.

3. The medical delivery device of claim 2, wherein the first actuator is configured to cause the pressurized fluid to agitate the agent, thereby forming a mixture of the agent and the pressurized fluid.

4. The medical delivery device of claim 3, further comprising a delivery conduit fluidly coupled to the body;
wherein the first actuator is configured to cause the pressurized fluid to move the mixture out of the body and into the delivery conduit.

5. The medical delivery device of claim 4, wherein the first actuator is configured to release the pressurized fluid from the fluid source at a first pressure level.

6. The medical delivery device of claim 5, further comprising a second actuator configured to release the pressurized fluid from the fluid source and into the body at a second pressure level that is less than the first pressure level.

7. The medical delivery device of claim 6, wherein the second actuator is configured to cause the pressurized fluid to inhibit a buildup of moisture within the delivery conduit without delivering the agent when the enclosure is in the first position.

8. The medical delivery device of claim 6, wherein the second actuator is configured to cause the pressurized fluid to flush the delivery conduit of a residual material without delivering the agent when the enclosure is in the first position.

9. The medical delivery device of claim 6, wherein the first actuator includes a trigger mechanism that is depressible relative to the body, and wherein the second actuator includes a lever mechanism that is pivotable relative to the body.

10. The medical delivery device of claim 1, wherein the body includes a handle having an interior cavity, and a cartridge configured to receive the fluid source;
wherein the cartridge is releasably coupled to the handle such that the fluid source is disposed within the interior cavity when the cartridge is coupled to the handle.

11. The medical delivery device of claim 10, wherein the cartridge includes a second actuator that is configured to move the fluid source within the interior cavity to fluidly couple the fluid source to the body.

12. The medical delivery device of claim 10, wherein the body includes a second actuator along the handle that is configured to move the fluid source within the interior cavity to fluidly couple the fluid source to the body.

13. The medical delivery device of claim 1, wherein the body includes a handle having an interior cavity, the handle is movable relative to the body from an open configuration to a closed configuration;
wherein, when in the open configuration, the handle is at least partially detached from the body and configured to receive the fluid source within the interior cavity, and in the closed configuration, the handle is attached to the body and configured to fluidly couple the fluid source to the body.

14. The medical delivery device of claim 1, further comprising a second actuator configured to move between a plurality of positions relative to the body to control an operating mode of the medical delivery device.

15. The medical delivery device of claim 14, wherein, in a first position of the plurality of positions, the second actuator is configured to control the medical delivery device in a neutral operating mode such that the pressurized fluid is not released from the fluid source upon actuation of the first actuator;
wherein, in a second position of the plurality of positions, the second actuator is configured to control the medical delivery device in a low pressure operating mode such that the pressurized fluid is released from the fluid source at a low pressure level upon actuation of the first actuator; and
wherein, in a third position of the plurality of positions, the second actuator is configured to control the medical delivery device in a high pressure operating mode such that the pressurized fluid is released from the fluid source at a high pressure level that is greater than the low pressure level upon actuation of the first actuator.

16. A medical delivery device, comprising:
a body including a handle, a central body, and a nozzle, the body is configured to be fluidly coupled to a fluid source storing a pressurized fluid;
an enclosure storing an agent; and
an actuator configured to release the pressurized fluid from the fluid source into the body;
wherein the enclosure is configured to move relative to the body from a first position, in which the enclosure is fluidly decoupled from the fluid source such that the pressurized fluid released into the body upon actuation of the actuator is inhibited from fluidly communicating with the agent stored in the enclosure, to a second position in which the enclosure is fluidly coupled to the fluid source such that the pressurized fluid released into the body upon actuation of the actuator fluidly communicates with the agent stored in the enclosure.

17. The medical delivery device of claim 16, wherein, when the enclosure is in the first position, the pressurized fluid released from the fluid source into the body is at a first pressure level; and
wherein, when the enclosure is in the second position, the pressurized fluid released from the fluid source into the body is at a second pressure level that is greater than the first pressure level.

18. The medical delivery device of claim 17, wherein the actuator is configured to cause the pressurized fluid at the first pressure level to inhibit a buildup of moisture within a delivery conduit coupled to the nozzle without delivering the agent out of the body when the enclosure is in the first position.

19. The medical delivery device of claim 17, wherein the actuator is configured to cause the pressurized fluid at the second pressure level to agitate the agent and form a mixture of the agent and the pressurized fluid for delivery out of the body when the enclosure is in the second position.

20. A method for delivering an agent from a medical delivery device that includes a body, a fluid source, an enclosure, and an actuator, the method comprising:
moving the enclosure to a first position relative to the body to fluidly decouple the enclosure from the fluid source;
actuating the actuator to release a pressurized fluid from the fluid source into the body, wherein the pressurized fluid is not in fluid communication with the agent stored in the enclosure when in the first position;
moving the enclosure to a second position relative to the body to fluidly couple the enclosure to the fluid source; and
actuating the actuator to release the pressurized fluid from the fluid source into the body, wherein the pressurized fluid is in fluid communication with the agent stored in the enclosure when in the second position;
wherein the pressurized fluid agitates the agent and forms a mixture of the agent and the pressurized fluid for delivery out of the medical delivery device.
